(19) United States
(12) Patent Application Publication
(10) Pub. No.: US 2023/0253118 A1
(43) Pub. Date: Aug. 10, 2023

(54) METHOD FOR CLASSIFYING A SUBJECT SUSPECTED TO SUFFER FROM AN ACUTE EVENT IN A RISK GROUP

(71) Applicants: SORBONNE UNIVERSITÉ, Paris (FR); INSERM (INSTITUT NATIONAL DE LA SANTÉ ET DE LA RECHERCHE MÉDICALE), Paris Cedex 13 (FR)

(72) Inventors: Delphine SAUCE, Saint-Maur-des-Fossés (FR); Martin LARSEN, Saint-Maur-des-Fossés (FR)

(73) Assignees: SORBONNE UNIVERSITÉ, Paris (FR); INSERM (INSTITUT NATIONAL DE LA SANTÉ ET DE LA RECHERCHE MÉDICALE), Paris Cedex 13 (FR)

(21) Appl. No.: 18/004,710

(22) PCT Filed: Jul. 12, 2021
(86) PCT No.: PCT/EP2021/069367

§ 371 Date: Jan. 9, 2023

(30) Foreign Application Priority Data
Jul. 10, 2020 (EP) 20305794.8

(51) Int. Cl. G16H 50/30; G01N 33/53

(52) U.S. Cl. CPC G16H 50/30; G01N 33/5308

(57) ABSTRACT

An in vitro method of classifying a subject suspected to suffer from an acute event including the steps of: determining neopterin concentration in a biological sample obtained from the subject, comparing the concentration with a predetermined reference neopterin concentration, and assigning the subject to a risk group based on the comparison of the neopterin concentration with the predetermined reference neopterin concentration.

### FIELD

The present application is in the field of diagnostic methods.

The application pertains generally to methods of classifying a subject suspected to suffer from an acute event such as a viral infection.

### BACKGROUND

The coronavirus disease 2019 (COVID-19), caused by the severe acute respiratory syndrome-coronavirus 2 (SARS-CoV-2) has emerged as a pandemic, claiming more than 500 000 deaths and around 12 million confirmed cases world-wide between December 2019 and June 2020. The disease caused by the virus, COVID-19, is characterized by none, mild or severe disease. The disease severity is tightly associated with age and the presence of comorbidities. Indeed, mortality rates are above 15% for the elderly (+80y). In response to the rising numbers of cases and deaths, and to maintain the capacity of health systems to treat as many severe cases as possible, most countries have implemented measures to control the pandemic and require tools for patients' severity stratification.

Since SARS-CoV-2 discovery and identification, the energy deployed by the scientific community has made it possible to generate an extraordinary amount of data, both in terms of quality and quantity. However, to date, clinically approved biomarkers are lacking.

Clinical decision-making regarding SARS-CoV2 is thus mainly based on medical parameters since no biomarkers can be used to decide on adapted care for SARS-CoV2 patients. Scores and tests have been proposed but they have not been clinically useful without external validation.

Consequently, there is a need in the art for simple, efficient, objective and reliable methods for classifying patients according to their risk level.

In this context, the Inventors have developed an innovative approach using neopterin to stratify the patients according to the estimated severity of their condition.

In the present application, the inventors show that neopterin also pinpoints high-risk patients since its concentration is predictive of the outcome of a viral infection in tested patients. No correlation was previously made between neopterin concentration and the outcome of such infection.

A first object of the present invention is hence an in vitro of classifying a subject suspected to suffer from an acute event. Such a method can be used at home, in a pharmacy, during a medical appointment, in the emergency ward and/or during the hospital stay.

A second object of the invention is a method of treatment, comprising the steps of the method for classifying a subject according to the first aspect of the invention and comprising an additional step of adapting the treatment.

Another object of the invention is a kit for classifying a subject suspected to suffer from an acute event.

Measuring the concentration of neopterin at arrival to hospital should enable to identify patients at risk of a severe disease course (high neopterin concentrations) and ensure that critical care beds and specialized hospital wards are dedicated to their treatment. In the case of limited ICU capacity, the concentration of neopterin will enable the selection of patients admitted or not in the ICU unit. In doing so healthcare resources will be put to best use and the pressure on specialized hospital wards will be reduced.

Previously, physicians most likely directed patients toward these units on a first come, first served basis, or with the help of inaccurate markers and/or their own expertise. As a consequence, patients which would benefit the most from these units are not preferentially directed to these.

### SUMMARY

This invention thus relates to an in vitro method of classifying a subject suspected to suffer from an acute event comprising the following steps: (i) determining neopterin concentration in a biological sample obtained from said subject; (ii) comparing said concentration with a predetermined reference neopterin concentration, and (iii) assigning the subject to a risk group based on the comparison of the neopterin concentration with the predetermined reference neopterin concentration.

In a particular aspect of this method the subject is assigned to a low-risk group if the neopterin concentration of the subject is lower than the predetermined reference neopterin concentration.

In a particular aspect of this method the subject is assigned to a high-risk group if the neopterin concentration of the subject is higher than the predetermined reference neopterin concentration.

According to a further aspect of the method, the predetermined reference neopterin concentration is superior to 10 nmol/L, preferably the predetermined reference neopterin concentration is comprised between 10 and 100 nmol/L, more preferably between 40 and 70 nmol/L.

In one embodiment, the predetermined reference neopterin concentration is 53 nmol/L.

In another embodiment, the predetermined reference neopterin concentration is 51 nmol/L.

The invention also related in a specific aspect to an in vitro method of classifying a subject suspected to suffer from an acute event wherein the high-risk group classified subjects suffering from a disease caused by an acute event, more particularly by a viral infection are at risk of having or developing a severe form and/or a complication of the disease caused by a viral infection or even at risk of death occurring after the virus infection.

The invention also related in a specific aspect to an in vitro method of classifying a subject suspected to suffer from an acute event wherein the subject is assigned to a no-risk group if the neopterin concentration is lower than a predetermined reference neopterin concentration.

According to the above aspect of the method of classifying a subject suspected to suffer from an acute event, the subject is assigned to a no-risk group if the predetermined reference neopterin concentration is comprised between 5 and 40 nmol/L, more preferably between 10 and 30 nmol/L, more preferably 15 and 25 nmol/L.

In one embodiment, the predetermined reference neopterin concentration is 19 nmol/L.

In another embodiment, the predetermined reference neopterin concentration is 20 nmol/L.

The present invention relates to in vitro methods, wherein the acute event is a viral infection, preferably a coronavirus infection, more preferably an infection by a coronavirus selected from the list consisting of SARS-CoV, MERS-CoV and SARS-CoV2.

As such, and in a specific aspect of the invention, the acute event is an infection by SARS-CoV2.

The present invention relates to in vitro methods, wherein the biological sample is selected from the group comprising a blood sample, a broncho-alveolar lavage sample, a serum sample, a plasma sample, a urine sample, a saliva sample, a cerebrospinal fluid sample and a feces sample.

According to a specific aspect, the determination of the neopterin concentration is performed by ELISA, mass spectrometry, high-performance liquid chromatography or Lateral Flow Immunoassay.

The invention also relates to a kit for classifying a subject suspected to suffer from an acute event. And more particularly, the kit comprises a collection mean for the sample, and the reagents necessary to carry out the method according to the invention.

### BRIEF DESCRIPTION OF THE DRAWINGS

FIG. 1A is a Receiver Operating Characteristics (ROC) curve depicting sensitivity and specificity of prediction model for SarsCov-2 infection based on neopterin concentration. Grey zone represents confidence interval. AUC=0.992; Specificity=100%; Sensitivity=97%; threshold:=20 nM.

FIG. 1B is a violinplot representing serum neopterin concentrations (in nM) stratified according to infectious status (CTRL (healthy donors, uninfected); Covid-19 (SarsCov-2 infected individuals) Shape of dots represents final clinical outcome in infected patients (Black circles for Recovery; Black triangles for non-survivors and long-term stay). Dotted line shows the threshold (20 nM) enabling the stratification of healthy vs infected patients.

FIG. 2A is a Receiver Operating Characteristics (ROC) curve depicting sensitivity and specificity of prediction model for fatal disease outcome based on neopterin concentration in early days of disease (less than 21 days). Grey zone represents confidence interval. AUC=0.967; Specificity=94%; Sensitivity=88%; threshold:=60 nM.

FIG. 2B is a violinplot representing serum neopterin concentrations (in nM) stratified according to disease severity and outcome (CTRL (healthy donors); DEAD (non-survivors); RECOVERY (survivors). Dotted line shows the threshold (60 nM) enabling the stratification of dead and survivors within 21 days post infection.

FIG. 3A is a Receiver Operating Characteristics (ROC) curve depicting sensitivity and specificity of prediction model for SarsCov-2 infection based on neopterin concentration. Grey zone represents confidence interval. AUC=0.963; Specificity=100%; Sensitivity=87%; threshold=19 nM.

FIG. 3B is a violinplot representing serum neopterin concentrations (in nM) stratified according to infectious status (CTRL (healthy donors, uninfected); Covid-19 (SarsCov-2 infected individuals) Shape of dots represents country of inclusion of infected patients (Circles for France; Triangles for Spain). Dotted line shows the threshold (19 nM) enabling the stratification of healthy vs infected patients.

FIG. 4A is a Receiver Operating Characteristics (ROC) curve depicting sensitivity and specificity of prediction model for fatal disease outcome based on neopterin concentration. Grey zone represents confidence interval. AUC=0.9; Specificity=64%; Sensitivity=100% ; threshold=53 nM.

FIG. 4B is a violinplot representing serum neopterin concentrations (in nM) stratified according to clinical outcome (RECOVERY (survivors) and DEAD (non-survivors); Dotted line shows the threshold (5 3nM) enabling the stratification of deceased and survivors.

FIG. 5 is a Kaplan-Meier estimator graph with log-rank based statistics featuring survival curves for Covid-19 patients with high (>53 nm) or low (<53 nM) serum concentration of neopterin at hospital admission.

FIG. 6A is a scatter plot graph representing the correlation between the concentration of neopterin measured at D0 (nmol/l) and the disease duration (days). r=0.23; p=0.0001.

FIG. 6B is a scatter plot graph representing the correlation between the concentration of neopterin measured at D9 (nmol/l) and the length of hospital stay (days). r=0.26; p<0.0001.

FIG. 7 is a scatter plot graph associating serum neopterin concentration (in nM) and age. Final outcome is represented by colored symbols (grey as recovery, black as non-survivors). Light grey symbols represent the 256 healthy volunteers. The shape of the symbols takes into account the hypertension status of the infected patient (no hypertension as circles, hypertension as triangles).

FIG. 8 is a scatter plot representing the correlation between the concentration of neopterin measured at D0 (Nm) and the time since symptom onset (days) for both survivors (in grey) and deceased patients (in black).

### DETAILED DESCRIPTION

“Acute event” refers to any infectious disease from which a subject suffers. As used herein, the term “infectious diseases” refers to diseases caused by pathogenic microorganisms, such as bacteria, viruses, parasites or fungi; the diseases can be spread, directly or indirectly, from one person to another. Such infectious diseases include, without limitation, infection by coronaviruses.
Neopterin refers to 2-amino-4-hydroxy-6-(D-erythro-, 2′, 3′-trihydroxypropyl) which has the following Formula I and belongs to the group of pteridines. Neopterin represents a precursor molecule of biopterin that is an essential cofactor in neurotransmitter synthesis, and it is also involved in a variety of oxydation/reduction reactions in the body. Neopterin is derived in vivo from guanosine triphosphate (GTP). The enzyme GTP-cyclohydrolase-I (GTPCH I) catalyses this reaction in activated monocytes, macrophages, dendritic cells, and endothelial cells and to a lesser extent in renal epithelial cells, fibroblasts, and vascular smooth muscle cells upon stimulation mainly by interferon gamma and to a lesser extent by interferon alpha and beta with its release being enhanced by tumor necrosis factor.

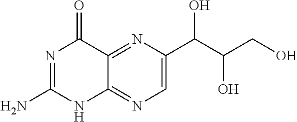 [SMILES: Nc1nc(=O)c2nc(C(O)C(O)CO)cnc2[nH]1]

A “sample” refers to sample obtained from a subject, for example blood, broncho-alveolar lavage, saliva, feces, urine, semen, blood plasma, synovial fluid or serum. In a particular embodiment, the biological sample is blood sample. The term “blood sample” means any blood sample derived from the subject. Typically, the concentration of neopterin is measured in a blood sample obtained when the subject arrives at hospital. In another embodiment, the blood sample is either fresh or cryo-preserved at −80° C. and the concentration of neopterin may be measured up to 36 months starting from the cryo-preservation. Typically, the serum has been obtained after collecting blood by venipuncture, allowing clotting. The clot is removed by centrifugation at room temperature and the resulting supernatant, designated serum, is carefully removed using Pasteur pipette. Plasma is produced when whole blood is collected into tubes that are treated with anticoagulant (such as heparine or EDTA).
“Risk classification”, as used herein, means a grouping of subjects by the level of risk (or likelihood) that the subject will experience a particular clinical outcome. A subject may be classified into a risk group or classified at a level of risk based on the methods of the present disclosure, e.g., high, medium or low risk. A “risk group” is a group of subjects or individuals with a similar level of risk for a particular clinical outcome.
A “fresh frozen” sample refers to a sample which has been frozen less than 48 hours after its collection.
A “subject” is a human, either male or female, adult or children.
“Enzyme-linked immunosorbent assays” (ELISA), refers to an assay using a solid-phase enzyme immunoassay to detect the presence of a ligand in a liquid sample using antibodies directed against the ligand to be measured. ELISA, in general, are performed by binding a reference reagent (antigen) to a solid phase support. Test sera, mixed with a labeled reagent, is then reacted with the bound reference reagent. The reagents are then subjected to a series of dilution, incubation, and washing steps in order to separate bound and free reagents. The process concludes with a detection step, compatible with the type of label used, designed to indirectly measure the amount of antibody (or antigen) in the test sera. A competitive ELISA, is performed by binding an anti-ligand antibody to a solid phase support. Test sera or serial dilutions of a standard with known ligand concentration are mixed with a labeled ligand. The mix is then reacted competitively with the bound antibody. The reagents are then subjected to incubation, and washing steps in order to separate bound and free ligands. The process concludes with a detection step, compatible with the type of label used, designed to indirectly measure the amount of ligand (or antigen) in the test sera.
“Mass spectrometry” refers to an analytical technique that measures the mass-to-charge ratio of ions to determine the masses of particles and of molecules in a sample. Mass spectrometry, whatever its type, generally includes steps consisting in identifying molecules present in a sample by measuring the mass of these molecules after they have been ionized, accelerated and introduced in a mass spectrometer.
“High-performance liquid chromatography” (HPLC) refers to a technique used in analytical chemistry to separate, identify, and quantify each component in a mixture. Typically, a pressurized liquid solvent containing the sample mixture is passed through a column filled with a solid adsorbent material. Each component in the sample interacts slightly differently with the adsorbent material, causing different flow rates for the different components and leading to the separation of the components as they flow out of the column
“Lateral Flow Immunoassay”, also known as lateral flow immunochromatographic assays, Lateral flow test or Lateral flow device, refers to devices intended to detect the presence of a target analyte in liquid sample by running said liquid along the surface of a pad comprising a series of capillary beds, such as pieces of porous paper, microstructured or nanostructured polymer, or sintered polymer with reactive molecules that show a visual positive or negative result.

The invention relates to a method for classifying a subject suspected to suffer from an acute event comprising the following steps:
i) determining neopterin concentration in a biological sample obtained from said subject;
ii) comparing said concentration with a predetermined reference neopterin concentration and
iii) assigning the subject to a risk group based on the comparison of the neopterin concentration with the predetermined reference neopterin concentration.

This method allows a physician to determine whether the subject is at risk and is therefore a subject in need of a specific care adapted to its situation, or is at low risk or no risk and is therefore a subject which will recover with a standard level of care or not suffer at all from any complication.

Consequently, a subject identified as being at risk will be monitored closely, its care regimen adapted to suit its need, or will be addressed to a specialized care unit, improving its survival chance, its recovery and its quality of life after the discharge from the hospital.

In one embodiment, the subject is assigned to a low-risk group if the neopterin concentration is lower than the predetermined reference neopterin concentration.

In one embodiment, the subject may be assigned to a low risk group. As used herein, the term “low risk group” refers to a group of subjects whose acute event is not likely to evolve to a higher stage of the disease, i.e. these subjects are diagnosed with a low risk viral infection. Such low risk viral infection does not cause extended harm to the subject, the infection being asymptomatic, paucisymptomatic or mildly symptomatic. In a low risk group, the symptoms are likely to be less severe, and includes headache, muscle pain, fatigue, tiredness, minor fever, anosmia and/or ageusia. In one embodiment, a lung scan of a subject from a low risk group does not show lung damage. The duration of hospital stay will be shorter compared to a subject assigned to a high-risk group. A subject assigned to a low-risk group is not a risk of dying from the acute event.

According to a further aspect of the method, the predetermined reference neopterin concentration is superior to 10 nmol/L, preferably the predetermined reference neopterin concentration is comprised between 10 and 100 nmol/L, more preferably between 40 and 70 nmol/L, more preferably the predetermined reference neopterin concentration is 49 nmol/L and even more preferably the predetermined reference neopterin concentration is 53 nmol/L.

In a more specific aspect, the subject is assigned to a low-risk group if the neopterin concentration of the subject is lower than the predetermined reference neopterin concentration which is 53 nmol/L.

In one embodiment, the subject is assigned to a low-risk group if the neopterin concentration of the subject is comprised between 20 nmol/L and 49 nmol/L.

In an even more specific aspect, the subject is assigned to a low-risk group if the neopterin concentration of the subject is comprised between 19 nmol/L and 53 nmol/L.

In one embodiment, the subject is assigned to a high-risk group if the neopterin concentration is higher than the predetermined reference neopterin concentration.

In one embodiment, the subject may be assigned to a high-risk group. As used herein, the term “high risk group” refers to a group of subjects whose acute event is likely to evolve to a higher stage of the disease. In a particular aspect, the acute event is a viral infection.

A viral infection is likely to cause harm to the subject, and curation would necessitate a medical treatment. In a high-risk group, the symptoms are likely to be more severe, and includes, additionally to the symptoms of the low risk group, severe fever, cough, chest pains, respiratory discomfort, pneumonia affecting one or both lungs and/or disorientation. In one embodiment, a lung scan of a subject from a high-risk group shows lung damage. The acute event of subject classified in the high-risk group is likely to evolve to a life-threatening stage of the disease. In such a stage, the disease is sure to cause extended harm to the subject, and would necessitate a heavy medical treatment to keep the subject alive.

A subject assigned to a high-risk group would typically be in need of one or more treatment, such as transfer to an intensive care unit, higher dosage regimen, combined use of drugs and/or being in a controlled environment. The discharge from hospital will be delayed compared to a subject assigned to a low risk group. In addition, a subject assigned to a high-risk group is more likely to die from said acute event.

In another embodiment, the predetermined reference neopterin concentration is superior to 10 nmol/L. Preferably, the predetermined reference neopterin concentration is comprised between 10 and 100 nmol/L, preferably 40 and 70 nmol/L. More preferably, the predetermined reference neopterin concentration is 49 nmol/L. Even more preferably the predetermined reference neopterin concentration is 53 nmol/L.

In an even more specific aspect, the subject is assigned to a high-risk group if the neopterin concentration of the subject is higher than the predetermined reference neopterin concentration which is 53 nmol/L.

In one embodiment, the subject is assigned to a no risk group if the neopterin concentration is lower than a predetermined reference neopterin concentration.

In one embodiment, the subject may be assigned to a no risk group. As used herein, the term “no risk group” refers to a group of subjects not suffering from an acute event.

In a more specific embodiment, the subject assigned to a no risk group refers to a group of subjects not suffering from a viral infection.

In another embodiment, the predetermined reference neopterin concentration is comprised between 5 and 40 nmol/L. Preferably, the predetermined reference neopterin concentration is comprised between 10 and 30 nmol/L, preferably 15 and 25 nmol/L. More preferably, the predetermined reference neopterin concentration is 20 nmol/L. Even more preferably, the predetermined reference neopterin concentration is 19 nmol/L

In an even more specific aspect, the subject is assigned to a no-risk group if the neopterin concentration of the subject is lower than the predetermined reference neopterin concentration which is 19 nmol/L.

In one embodiment, the acute event is a viral infection, preferably a coronavirus infection, more preferably an infection by a coronavirus selected from the list consisting of SARS-CoV, MERS-CoV and SARS-CoV2.

In another embodiment, the acute event is an infection by SARS-CoV2.

In one embodiment, the subject of the method of the invention is a human subject, either male or female. In another embodiment, the subject has been diagnosed with a viral infection. In another embodiment, the subject suffers from at least one comorbidity. In a preferred embodiment, the comorbidity is selected from the list comprising obesity, diabetes, cardiovascular disease, pulmonary disease and/or smoking.

Surprisingly, the inventors found that neopterin concentration is not associated with comorbidities such as age, hypertension, diabetes and cardiovascular disease. Hence, neopterin concentration allows physician to assess the risk group of a patient independently of comorbidity status, which limits the problem caused by an undiscovered comorbidity or by a patient unable to communicate about his or her state.

In a preferred embodiment, the biological sample is selected from the group comprising a blood sample, a broncho-alveolar lavage sample, a serum sample, a plasma sample, a urine sample, a saliva sample, a cerebrospinal fluid sample and a feces sample.

In a preferred embodiment, the biological sample is fresh, fresh frozen or frozen.

In a preferred embodiment, said biological sample is obtained at a time chosen from the group comprising:
a) Following medical management,
b) At hospital arrival,
c) At inter-hospital transfer,
d) At intra-hospital transfer.

In a preferred embodiment, said biological sample is obtained at hospital arrival.

Methods for determining a neopterin concentration in a biological sample are well known in the art. Examples of such methods include, but are not limited to, immunohistochemistry, multiplex methods (Luminex), enzyme-linked immunosorbent assay (ELISA), sandwich ELISA, fluorescent-linked immunosorbent assay (FLISA), enzyme immunoassay (EIA), radioimmunoassay (RIA), mass spectrometry (MS), a microarray, and the like, high-performance liquid chromatography or Lateral Flow Immunoassay, or any combination thereof.

In a preferred embodiment, step i) the method of the invention is performed by ELISA, mass spectrometry, high-performance liquid chromatography or Lateral Flow Immunoassay.

In one embodiment, the methods of the invention comprise a step of assigning the subject to a risk group based on the comparison of the neopterin concentration with the predetermined reference neopterin concentration or the predetermined reference neopterin concentration.

In a particular embodiment the neopterin concentration is measured by competitive enzyme-linked immunosorbent assay (ELISA) by following commercial kits (e.g. DRG Diagnostics, Biomnis) using horseradish peroxidase (HRPO)-labeled neopterin. Particularly, ELISA is suitable for measuring neopterin concentration in saliva, urine and blood sample.

In another embodiment the neopterin concentration is assayed by high pressure liquid chromatography (HPLC; see, e.g., Huber et al., J. Chromatography B: Biomed. Sci. App. 666(2): 223-232 (April 1995) regarding HPLC of neopterin in serum) with fluorescence detection after appropriate sample clean-up. Particularly, HPLC is performed to measure neopterin concentration in urine or plasma samples.

In one embodiment, the conclusion regarding the prognosis of the subject is taken after comparing the neopterin concentration of a specific sample of a subject following an acute event, preferably a body fluid sample, more preferably a blood or serum sample to a predetermined reference value.

In an embodiment, the person skilled in the art define a predetermined reference neopterin concentration with a given method of measurement. In this embodiment, the predetermined reference neopterin concentration is the neopterin concentration obtained from a control sample or a reference population. In this embodiment, the neopterin concentration is determined with the same method of measurement as the predetermined reference neopterin concentration. Therefore, the person skilled in the art knows how to measure the neopterin concentration in a subject using a given method of measurement and to compare said neopterin concentration with a predetermined reference neopterin concentration obtained from a control sample or from a reference population to classify the subject in a risk group depending on the concentration of neopterin measured.

Typically, a reference neopterin concentration may be either implemented in a software or an overall median or other arithmetic mean across measurements may be built.

In one embodiment, the predetermined reference neopterin concentration is derived from the measurement of the neopterin concentration according to the invention, in a control sample derived from a reference population.

In one embodiment, the reference population includes without limitation, such subjects having similar age range, the same sex, subjects in the same or similar ethnic group, and the like.

In one embodiment, the reference population comprises subjects, preferably at least 50, more preferably at least 100, more preferably at least 200 and even more preferably at least 500 subjects.

In one embodiment, the reference population comprises substantially healthy subject(s).

As used herein, a “substantially healthy subject” has not previously suffered from an acute event. In a preferred embodiment, a substantially healthy subject has not suffered from an acute event during the three months before collection of said control sample. In a preferred embodiment, a substantially healthy subject is not treated with an immune system altering treatment.

In one embodiment, the reference population comprises subjects suspected to suffer from an acute event. In a preferred embodiment, the reference population comprises subjects suffering from the same acute event as the subject to be treated.

In another embodiment, the reference population comprises samples obtained at hospital arrival from subjects that were suffering from the same acute event as the subject to be treated, and which were discharged from the hospital two weeks or less after their hospital arrival.

In another embodiment, the reference population comprises subjects suffering from the same comorbidity as the subject to be treated. Such comorbidity is selected from the list comprising hypertension, obesity, diabetes, cardiovascular disease, chronic respiratory disease and/or smoking.

The invention also relates to a method for predicting the prognosis of a subject suffering from an acute event comprising the following steps:
i) determining neopterin concentration in a biological sample obtained from said subject;
ii) comparing said concentration with a predetermined reference neopterin concentration and
iii) assigning the subject to a risk group based on the comparison of the neopterin concentration with the predetermined reference neopterin concentration.

The invention also relates to a method of treatment, of a subject suspected to suffer from an acute event comprising the following steps:
(i) determining neopterin concentration in a biological sample obtained from said subject;
(ii) comparing said concentration with a predetermined reference neopterin concentration and
(iii) assigning the subject to a risk group based on the comparison of the neopterin concentration with the predetermined reference neopterin concentration.
(iv) adapting the treatment of the subject according to the prediction of step iii.

In a preferred embodiment, step iv) comprises providing the subject with extracorporeal life support, such as mechanical ventilatory support or extracorporeal membrane oxygenation (ECMO). Alternatively, step iv) comprises directing the subject to an intensive care unit. Alternatively, step iv) comprises directing the subject to a classical hospital bed. Alternatively, step iv) comprises directing the subject to home quarantine and remote follow-up.

The invention also relates to a kit for classifying a subject suspected to suffer from an acute event.

In an embodiment of the invention, the kit comprises a collection mean for the sample, as well as the reagents necessary to carry out the method according to the first aspect of the invention.

The invention also relates to a Lateral Flow Immunoassay (LFI) device. The device comprises a sample pad—i.e. an area comprising a series of capillary beds, such as pieces of porous paper, microstructured polymer, or sintered polymer—and a conjugate pad in which the manufacturer has stored bio-active particles called conjugates in a matrix. The conjugate pad contains all the reagents required for an optimized chemical reaction between the target molecule (e.g., an antigen) and its chemical partner (e.g., antibody) that has been immobilized on the particle's surface. This marks target particles as they pass through the pad and continue across to the test and control lines. The test line shows a signal, often a color as in pregnancy tests. The test may also be quantitative and indicate a concentration of the target molecule.

Other advantages and features of the present invention will become readily apparent from the following detailed description of the invention. The following figures and examples are put forth so as to provide those of ordinary skill in the art with a complete disclosure and description of how to make and use the present invention, and are not intended to limit the scope of what the Inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EXAMPLE

The present invention is further illustrated by the following example.

In a first cohort, more than 100 individuals suffering from Covid-19 infection were screened to participate in our study, from either emergency department, either ICU, either classical hospital ward. Samples from around 30 healthy volunteers were obtained from EFS, a blood bank.

Sera have been cryopreserved until use.

In a second cohort, aA total of 610 individuals took part in this case-control design study including 374 participants with diagnosis of Covid-19 according to WHO criteria and positive SARS-CoV-2 RT-PCR testing on a respiratory sample (nasopharyngeal swab or invasive respiratory sample) as well as 256 age and sex-matched healthy controls. In France SARS-CoV-2-infected patients (n=65) was admitted either to the Department of Emergency, Intensive Care Unit (ICU) or to the Internal Medicine ward at Pitié-Salpêtriére Hospital (Paris, France) for symptomatic SARS-CoV-2 viral infection between Mar. 17 and May 11, 2020. Serum separating tube from blood samples were collected at admission (day of arrival to hospital). In Spain, SARS-CoV-2 infected individuals (n=309) were recruited for Germans Trias i Pujol Hospital, Institut Català de la Salut (Barcelona, Spain) after admission in either the Department of Emergency, ICU or to the Pneumology ward between Mar. 15 to Apr. 11, 2020. For each individual, sera were cryopreserved at −80° C. until use. The present study consists of SARS-CoV-2 infected patients with known clinical outcome within three months post hospital admission.

Data collected prospectively included age, sex, previous medical history, date of symptoms onset, and duration of hospitalization. Associated comorbidities were carefully reviewed, with a particular focus on the history of cardiovascular or respiratory events, diabetes and obesity.

The inventors chose to focus on the concentration of neopterin reached by the patients at their arrival to hospital.

Neopterin was analyzed as a predictive marker of prognosis of SARS-COV2, according to the following protocol.

### Material and Methods

Experiments were performed using a commercially available kit from Tecan by following manufacturer's instructions (described below). All samples were centrally processed and analyzed in Cimi-Paris.

### Principle of the Test

Neopterin ELISA kit is based on competitive binding of human Neopterin from serum samples and enzyme-labeled Neopterin to Neopterin specific antibodies immobilized on microtiter plates. After a washing step, chromogenic substrate is added and color developed. The enzymatic reaction (blue color) is inversely proportional to the amount of Neopterin present in the sample. The reaction is terminated by adding stopping solution (converts blue to yellow). Absorbance is then measured on an ELISA reader at 450 nm and the concentration of Neopterin in samples and control is read off the standard curve.

### Sensitivity

The lower detection limit is calculated from the standard curve by determining the resulting concentration of the mean OD of Calibrator A (based on 10 replicate analyses) minus 2 SD. The sensitivity of the Neopterin ELISA kit is 0.7 nmol/L.

### Specificity (Cross Reactivity)

The following compounds were tested for cross-reactivity with the Direct Neopterin ELISA kit. No significant interference was detected at the following concentration. Hemoglobin 35 mg/dL, Bilirubin 2.25 mg/dL, Triglyceride 125 mg/dL.

### Specimen Collection and Handling

Blood was collected by venipuncture, allowed to clot, and serum was separated by centrifugation at room temperature. The serum was not heat inactivate. If sera cannot be immediately assayed, they could be stored at −20° C. for up to six months. While the sample can be frozen and thawed, repeated freezing and thawing of samples should be avoided. Specimens containing NaN3 were not used. Samples appearing turbid were centrifuged before testing to remove any particulate material.

Experiments were performed on frozen human sera. The sera samples have been aliquoted, protected from light and cryopreserved at −80° C. before usage. Thawing has been done by putting samples in a fridge allowing slow thawing. Thereafter, thawed sera have been centrifuged at 1000 rpm during 10 min in the dark.

Reagents Preparation Stock Wash buffer was diluted (1:20) with water and stored at 4° C. for 1 month. All reagents were at room temperature prior to their use.

For the following experiment, human sera aliquots were used undiluted and manipulated in the dark. If necessary, samples to be used would have been diluted with assay buffer (1:10).

### Storage and Stability

The microtiter well plate and all other reagents are stable at 2-8° C. until the expiration date printed on the label. The whole kit stability is usually 6 months from the date of shipping under appropriate storage conditions. The unused portions of the standards should be stored at 2-8° C. or stored frozen in small aliquots.

### Test Procedure

All reagents were allowed to reach room temperature before use. The required number of coated strips were removed and arranged on the microtiter well plate.

The microtiter well strips to be used on the plate were labeled and the wash buffer was diluted with water (1:20).

20 μl of standards, controls, and samples were pipeted into appropriate wells in duplicate.

100 μl of ready-to-use enzyme conjugate were added into each well, followed by 50 μl of ready-to-use Neopterin antiserum into each well, before gently mixing for 5-10 seconds. The plate was covered and incubated for 90 minutes at 18-25° C. on orbital shaker (500 rpm) in the dark.

The well contents were aspirated and the plate blotted on absorbent paper before immediately washing the wells 4 times with 300 μl of 1× wash buffer.

150 μl of TMB substrate solution was added before gently mixing for 5-10 seconds. The plate was covered and incubated for 10 mins in the dark at 18-25° C.

The reaction was stopped by adding 150 μl of stop solution to all wells at the same timed intervals as in step 5. Gentle mixing was performed for 5-10 seconds to have uniform color distribution (blue color turned yellow).

Absorbance was measured at 450 nm using an ELISA reader within 15 min.

### Calculation of Results

The absorbance of all duplicates was averaged before subtracting the averaged non-specific binding (NSB) absorbance from the average obtained above. This yields the net absorbance.

### Formula:

Abs. (sample)−Abs. (NSB)

Abs.=average absorbance of duplicate wells

NSB=non-specific binding (also known as the blank) Sample=particular serum or standard being calculated Zero Standard=0 nmol/L standard

Construct a plot of the net absorbance (Y-axis) versus the concentration of the neopterin standards ((X-axis) starting with the 0.5 nmol/L point. This yields the standard curve.

Using the standard curve, the neopterin concentration of each sample was determined.

### Expected Reference Neopterin Concentration

Usually, healthy subjects show the following values: healthy subjects neopterin concentration (Normal): <10 nmol/L (0.3-3.0 ng/mL). Conversion: Neopterin (nmol/L)×0.253 =ng/mL

### Statistical Analysis

Groups were compared using a parametric t-test: Paired comparisons were conducted with non-parametric Wilcoxon test. Differences in survival were analyzed with the Kaplan-Meier method, data on surviving or non-surviving patients were censored at three months post arrival to hospital. Survival curves were compared with a log-rank test. A Cox proportional hazard model was used to compute hazard ratios and associated 95% confidence intervals (CI). P values<0.05 were considered statistically significant. To quantify the accuracy of our predictive model we plotted receiver operating characteristic (ROC) curves, which depicts the specificity and sensitivity of the prediction variable and indicates the overall quality of the prediction model through the area under the ROC curve (AUC).

All statistical analysis was performed with GraphPad prism software and R studio (v1.4). Graphical representations were performed with the ggplot2 package (v3.3). ROC analysis was conducted with the pROC package (v1.17) and survival models were established with the Survival package (v3.2).

### Results & Discussion

### A—First Cohort

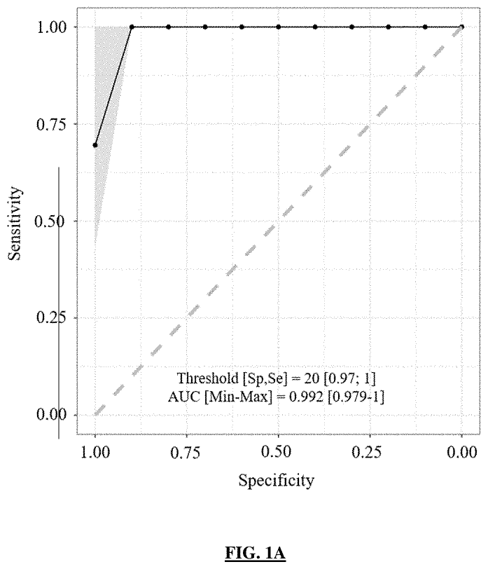

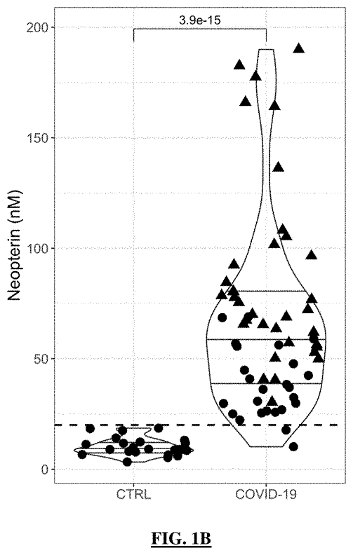

According to the concentration of neopterin measured in each samples the probability to discriminate between infected vs uninfected patients was statistically significant with 100% specificity and 97% sensitivity (AUC=0.992) (FIG. 1A).

As shown in FIG. 1B, the concentration of neopterin at day 0 was different between the control group of healthy volunteers and the SARS-Cov2 infected patients (p=3.10⁻¹⁵) A cut-off value was set at 20 nmol/l, which corresponds to optimal threshold obtained by Youden's J statistic.

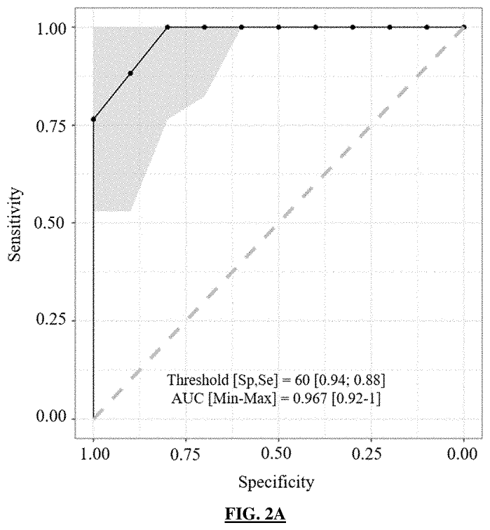

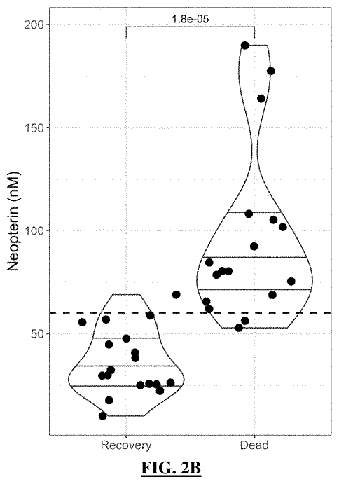

According to the concentration of neopterin measured in each patient at their arrival to the ward from which they were recruited, the probability of survival within 25 days post symptoms onset was statistically significant with 94% specificity and 88% sensitivity (AUC=0.967). (FIG. 2A).

As shown in FIG. 2B, the concentration of neopterin at day 0 was different between SARS-Cov2 infected patients who died compared to SARS-Cov2 infected survivors by taking into account early clinical outcome (within 25 days post symptoms onset) (p=1.8 10⁻⁵).

B—Second cohort The inventors demonstrate that neopterin can serve as a predictive biomarker of mortality post SARS-CoV-2 infection, enabling discrimination between patients who will recover and those at risk of fatal clinical outcome. Of note, neopterin level was measured at arrival to hospital before any treatment could interfere.

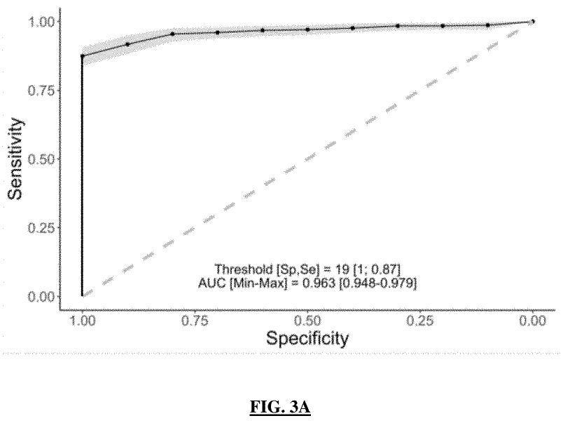

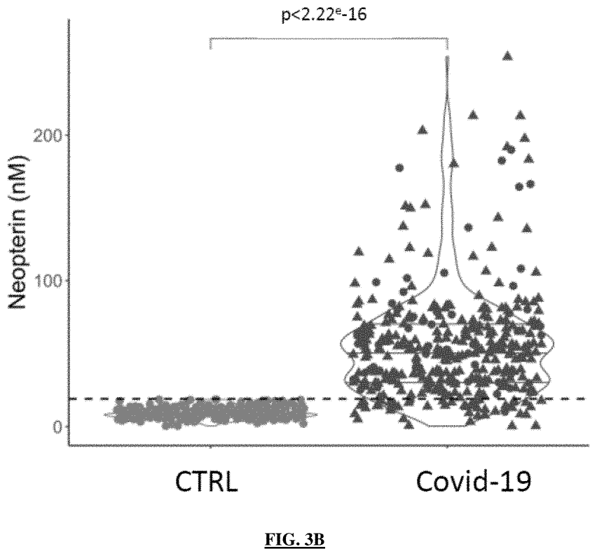

According to the concentration of neopterin measured in each sample at the patient's arrival to hospital, the probability to discriminate between infected vs uninfected patients was statistically significant with 100% specificity and 87% sensitivity (AUC=0.963) with a cut-off value set at 19 nmol/L (FIG. 3A).

As shown in FIG. 3B, the concentration of neopterin at day 0 was different between the control group of healthy volunteers and the SARS-Cov2 infected patients (9.5nM vs 56 nM; p=2.10-16).

A cut-off value was set at 19 nmol/l which corresponds to optimal threshold obtained by Youden's J statistic.

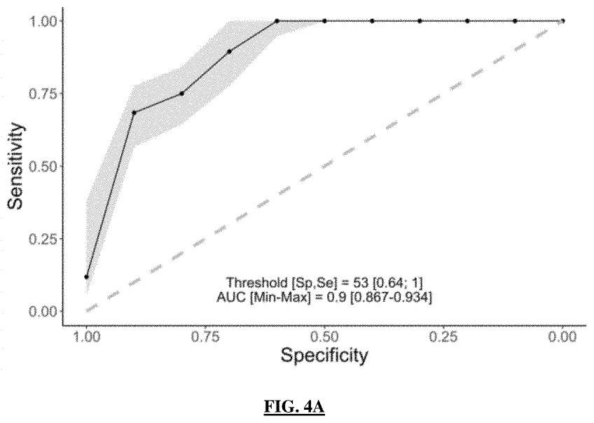

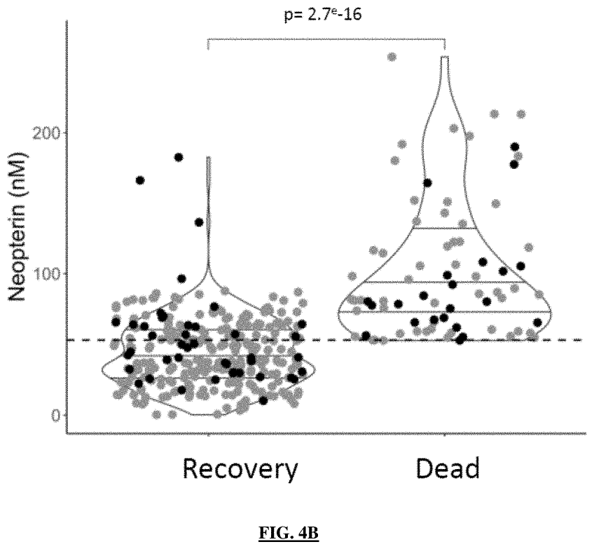

Therefore, to evaluate patients' clinical course, we estimate the predictive potential of elevated neopterin levels. According to the level of neopterin measured in each patient at their arrival to hospital, the probability of survival was significantly reduced for individuals with high systemic neopterin levels (FIG. 4A; AUC=0.9). The test has 64% specificity and 100% sensitivity.

A cut-off value was set at 53 nmol/l, which corresponds to optimal threshold obtained by Youden's J statistic.

As shown in FIG. 4B, the concentration of neopterin at admission was different between SARS-Cov2 infected patients who died compared to SARS-Cov2 infected survivors by taking into account early clinical outcome (101 nM vs 44nM; p=2.7×10-16).

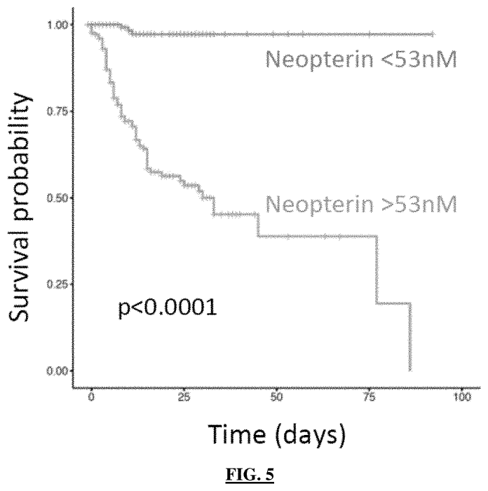

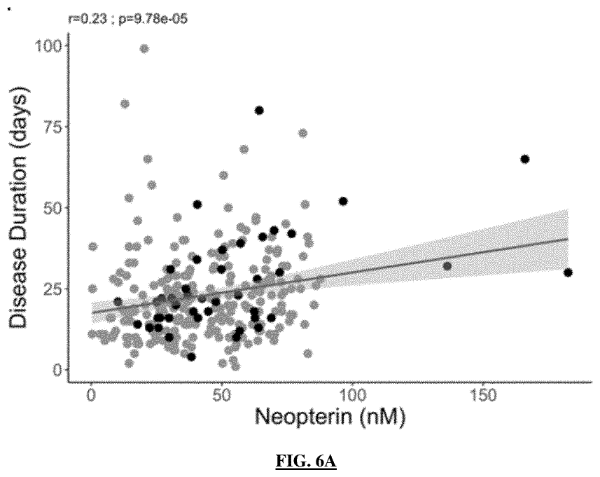

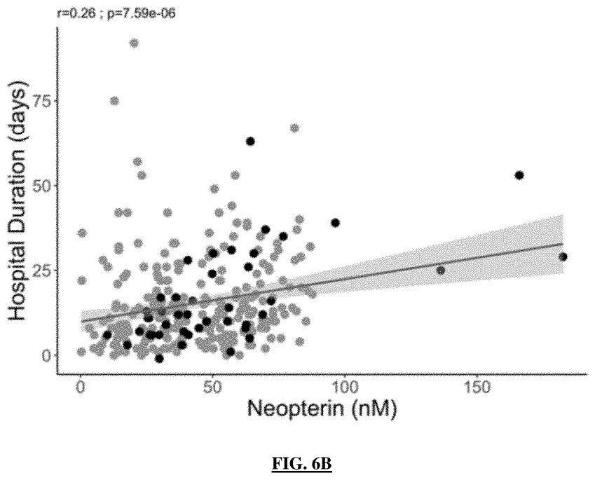

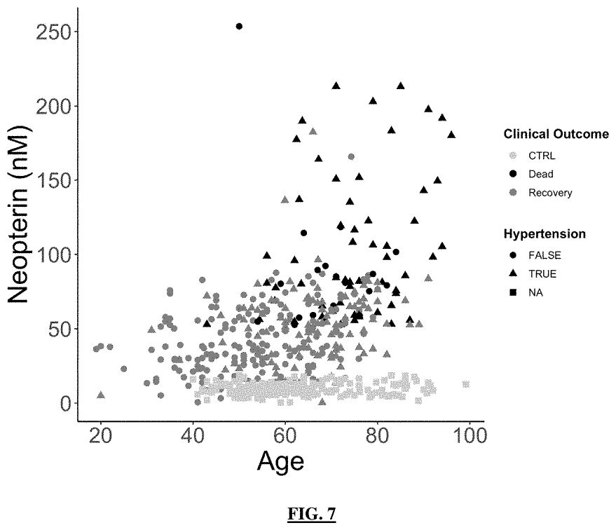

Based on 53 nM as threshold value, Kaplan-Meier survival curves were different between patients with low and high levels of systemic neopterin (FIG. 5; p<0.0001). As shown in FIG. 6, the concentration of neopterin at day 0 was correlated with both the disease duration (A) and the duration of hospital stay (B) demonstrating that neopterin help the stratification of patients at higher risk of pernicious outcome. Hypertension and in particular age appear to be risk-factors of non-survival (HR 0.49, 2.03 and 1.04; p=0.02, 0.03 and <0.001, respectively). We then assessed if those variables were correlated with neopterin levels. As shown, neopterin was not associated with age and hypertension status (FIG. 7). Of note, although high neopterin was found primarily in old age and individuals suffering from hypertension, the inverse was not true. Indeed, many individuals of old age or suffering from hypertension did not display high levels of neopterin nor did they die from SARS-CoV-2 infection. Therefore, neopterin level helps physicians to predict the outcome of SARS-CoV-2 infection in a patient, independently from comorbidities.

Here, we measured neopterin in patients' serum which results in an accurate estimation of disease and hence prognosis; however, this molecule is biologically and chemically stable in all body fluids.

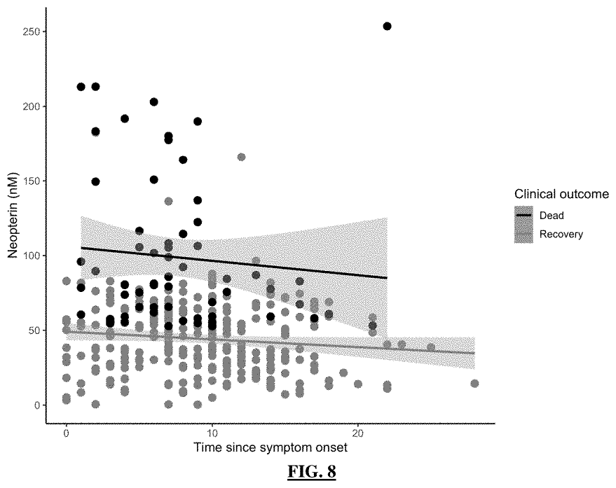

Importantly it is necessary to ensure that the marker would work irrespective of the time of arrival to hospital. Patient associated traits, such as anxiety, will impact on the delay between symptom onset and hospitalization. To rule out this problem we correlated neopterin levels and the time since symptom onset (FIG. 8). Although patients with fatal outcome were generally hospitalized fairly rapidly after symptom onset no general pattern would suggest that the biomarker would be affected by the time of analysis. Indeed, neopterin levels did not correlate with the time since symptom onset neither for patients with fatal outcome nor survivors.

Non-invasive salivatory or urinary measurements could also constitute a reliable assay to predict clinical outcome in this context for rapid testing.

In doing so, healthcare resources will be put to best use during pandemic peak periods and the pressure on specialized hospital wards will be reduced. Infected patients with low levels (<53 nM) of neopterin would be considered at low risk and can return to home quarantine and remote follow-up.

1-13. (canceled)

14. An in vitro method of classifying a subject suspected to suffer from an acute event comprising the following steps:
(i) determining neopterin concentration in a biological sample obtained from said subject;
(ii) comparing said concentration with a predetermined reference neopterin concentration; and
(iii) assigning the subject to a risk group based on the comparison of the neopterin concentration with the predetermined reference neopterin concentration.

15. The in vitro method of classifying a subject according to claim 14, wherein the subject is assigned to a low-risk group if the neopterin concentration of the subject is lower than the predetermined reference neopterin concentration.

16. The in vitro method of classifying a subject according to claim 15, wherein the predetermined reference neopterin concentration is superior to 10 nmol/L.

17. The in vitro method of classifying a subject according to claim 14, wherein the subject is assigned to a high-risk group if the neopterin concentration of the subject is higher than the predetermined reference neopterin concentration.

18. The in vitro method of classifying a subject according to claim 17, wherein the predetermined reference neopterin concentration is superior to 10 nmol/L.

19. The in vitro method of claim 17, wherein a high-risk group classified subject suffering from a disease caused by a viral infection is at risk of having or developing a severe form and/or a complication of the disease caused by a viral infection or at risk of death occurring after the virus infection.

20. The in vitro method according to claim 14, wherein the subject is assigned to a no risk group if the neopterin concentration is lower than a predetermined reference threshold neopterin concentration.

21. The in vitro method according to claim 20, wherein the predetermined reference threshold neopterin concentration is comprised between 5 and 40 nmol/L.

22. The in vitro method according to claim 14, wherein the acute event is a viral infection.

23. The in vitro method according to claim 22, wherein the acute event is a coronavirus infection.

24. The in vitro method according to claim 23, wherein the acute event is an infection by a coronavirus selected from the list consisting of SARS-CoV, MERS-CoV and SARS-CoV2.

25. The in vitro method according to claim 14, wherein the biological sample is selected from the group comprising a blood sample, a broncho-alveolar lavage sample, a serum sample, a plasma sample, a urine sample, a saliva sample, a cerebrospinal fluid sample and a feces sample.

26. The method according to claim 14, wherein step i) is performed by ELISA, mass spectrometry, high-performance liquid chromatography or Lateral Flow Immunoassay.

27. A kit for classifying a subject suspected to suffer from an acute event.

28. The kit of claim 17, comprising:
a collection mean for the sample, and
the reagents necessary to carry out the method according to claim 14.

29. A method of treatment, of a subject suspected to suffer from an acute event comprising the following steps:
(i) determining neopterin concentration in a biological sample obtained from said subject;
(ii) comparing said concentration with a predetermined reference neopterin concentration and
(iii) assigning the subject to a risk group based on the comparison of the neopterin concentration with the predetermined reference neopterin concentration.
(iv) adapting the treatment of the subject according to the prediction of step (iii).

30. The method of claim 29, wherein step (iv) comprises providing the subject with extracorporeal life support, such as mechanical ventilatory support or extracorporeal membrane oxygenation (ECMO).

31. The method of claim 29, wherein step (iv) comprises directing the subject to an intensive care unit.

32. The method of claim 29, wherein step (iv) comprises directing the subject to a classical hospital bed.

33. The method of claim 29, wherein step (iv) comprises directing the subject to home quarantine and remote follow-up.
